(19) United States
(12) Patent Application Publication
(10) Pub. No.: US 2010/0144696 A1
(43) Pub. Date: Jun. 10, 2010

(54) Stabilized pharmaceutical compositions containing a calcium channel blocker

(75) Inventors: Naoki Wakiyama, Yokohama-shi (JP); Fusao Usui, Yokohama-shi (JP); Kenji Nishimura, Yokohama-shi (JP)

Correspondence Address: FRISHAUF, HOLTZ, GOODMAN & CHICK, PC, 220 Fifth Avenue, 16TH Floor, NEW YORK, NY, 10001-7708, US

(73) Assignees: SANKYO COMPANY, LIMITED, Tokyo (JP); UBE INDUSTRIES, LTD., Tokyo (JP)

(21) Appl. No.: 12/658,201

(22) Filed: Feb. 3, 2010

Related U.S. Application Data
(63) Continuation of application No. 11/704,122, filed on Feb. 8, 2007.
Application No. 11/704,122 is a continuation of application No. 10/268,308, filed on Oct. 10, 2002, now abandoned.
Application No. 10/268,308 is a continuation-in-part of application No. PCT/JP01/03087, filed on Apr. 10, 2001.

(30) Foreign Application Priority Data
Apr. 11, 2000 (JP) 2000-108850

(51) Int. Cl. A61K 31/397; A61P 9/12

(52) U.S. Cl. 514/210.02

(57) ABSTRACT

The present invention provides a pharmaceutical composition containing a calcium channel blocker of the following formula or a pharmacologically acceptable salt thereof and a pharmacologically acceptable alkaline material which is added to an extent such that an aqueous solution or dispersion solution of said pharmaceutical composition containing a calcium channel blocker has a pH of at least 8:

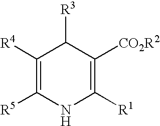

[wherein R¹ represents an optionally substituted C₁-C₄ alkyl group, an amino group or a cyano group; R² represents an optionally substituted C₁-C₄ alkyl group, a substituted C₃-C₄ alkenyl group, or a substituted 4- to 6-membered cyclic amino group; R³ represents a substituted phenyl group; R⁴ represents an optionally substituted C₁-C₄ alkoxycarbonyl group, a 1,3,2-phosphorinan-2-yl group, or a 5,5-dimethyl-1,3,2-phosphorinan-2-yl group, R⁵ represents a C₁-C₄ alkyl group].

This application is a continuation of application Ser. No. 11/704,122 (now pending) which is a continuation of application Ser. No. 10/268,308 filed Oct. 10, 2002 (now pending) which is a continuation-in-part application of International Application No. PCT/JP01/03087 filed Apr. 10, 2001, which is incorporated herein in its entirety by this reference.

### BACKGROUND OF THE INVENTION

The present invention relates to a stabilized pharmaceutical composition containing a calcium channel blocker.

1. Description of Related Art

Calcium blockers (calcium channel blockers) are well known as antihypertensive agents, which can exist in a lot of formulations and are commercially available (for example, U.S. Pat. No. 3,485,847, U.S. Pat. No. 3,985,758, U.S. Pat. No. 4,572,909 and the hike). These formulations, however, are not always satisfactory in their stability such as their storage stability. A pharmaceutical composition having excellent stability such as storage stability has been desired.

2. Brief Description of the Invention

The inventors have made a great effort on the study of pharmaceutical compositions containing calcium channel blockers for a long period. They have found that a pharmacologically acceptable alkaline material is added to a calcium channel blocker to afford a pharmaceutical composition having excellent stability such as storage stability.

The present invention relates to a stabilized pharmaceutical composition containing a calcium channel blocker.

### DETAILED DESCRIPTION OF THE INVENTION

The present invention is a pharmaceutical composition containing a calcium channel blocker of the following formula or a pharmacologically acceptable salt thereof and a pharmacologically acceptable alkaline material which is added to an extent such that an aqueous solution or dispersion solution of said pharmaceutical composition containing a calcium channel blocker has a pH of at least 8:

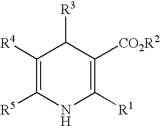

[wherein R¹ represents a C₁-C₄ alkyl group optionally substituted with carbamoyloxy or 2-aminoethoxy, an amine group or a cyano group,

R² represents a C₁-C₄ alkyl group optionally substituted with acetyl, N-methyl-N-(phenylmethyl optionally substituted with fluoro)amino, N-(phenyl optionally substituted with fluoro)-N-(phenylmethyl optionally substituted with fluoro)amino, 2-tetrahydrofuryl, or 4-[phenylmethyl optionally substituted with fluoro or di-(phenyl optionally substituted with fluoro)methyl]-1-piperazinyl, a C₃-C₄ alkenyl group substituted with phenyl in which said phenyl group is optionally substituted with fluoro, or a 4- to 6-membered cyclic amino group in which the nitrogen atom thereof is substituted with phenylmethyl optionally substituted with fluoro, or di-(phenyl optionally substituted with fluoro)methyl,

R³ represents a phenyl group which is substituted with 1 or 2 substituents selected from the group consisting of halogen, nitro and 1,2-methylenedioxy,

R⁴ represents a C₁-C₄ alkoxycarbonyl group optionally substituted with methoxy, a 1,3,2-phosphorinan-2-yl group, or 5,5-dimethyl-1,3,2-phosphorinan-2-yl,

R⁵ represents a C₁-C₄ alkyl group].

In formula (I):

The C₁-C₄ alkyl moiety of the C₁-C₄ alkyl group optionally substituted with carbamoyloxy or 2-aminoethoxy in the definition of R¹, the C₁-C₄ alkyl moiety of the C₁-C₄ alkyl group optionally substituted with acetyl, N-methyl-N-(phenylmethyl optionally substituted with fluoro)amino, N-(phenyl optionally substituted with fluoro)-N-(phenylmethyl optionally substituted with fluoro)amino, 2-tetrahydrofuryl, or 4-[phenylmethyl optionally substituted with fluoro or di-(phenyl optionally substituted with fluoro)methyl]-1-piperazinyl in the definition of R², the C₁-C₄ alkyl moiety of the C₁-C₄ alkoxycarbonyl group optionally substituted with methoxy in the definition of R⁴, and the C₁-C₄ alkyl moiety in the definition of R⁵ each are, for example, methyl, ethyl, propyl, isopropyl, butyl, isobutyl, s-butyl, or t-butyl. R¹ and R⁵ each are preferably a methyl or ethyl group, more preferably a methyl group. R² is preferably a methyl, ethyl, isopropyl, or isobutyl group. R⁴ is preferably a methyl, ethyl or isopropyl group.

The C₃-C₄ alkenyl group substituted with phenyl in which said phenyl group is optionally substituted with fluoro in the definition of R² may be, for example, a 3-phenyl-2-propenyl group, a 3-(4-fluorophenyl)-2-propenyl group, a 4-phenyl-3-butenyl group, or a 2-methyl-3-phenyl-2-propenyl group, and preferably a 3-phenyl-2-propenyl group.

The 4- to 6-membered cyclic amino group in which the nitrogen atom thereof is substituted with phenylmethyl optionally substituted with fluoro, or di-(phenyl optionally substituted with fluoro)methyl in the definition of R² may be, for example, a 1-benzyl-3-azetidinyl, 1-diphenylmethyl-3-azetidinyl, 1-(di-4-fluorophenylmethyl)-3-azetidinyl, 1-benzyl-3-pyrrolidinyl, 1-(4-fluorophenylmethyl)-3-pyrrolidinyl, 1-diphenylmethyl-3-pyrrolidinyl, 1-benzyl-3-piperidinyl, 1-(4-fluorophenylmethyl)-3-piperidinyl, or 1-diphenylmethyl-3-piperidinyl group, preferably a 1-benzyl-3-azetidinyl, 1-diphenylmethyl-3-azetidinyl, 1-benzyl-3-pyrrolidinyl, or 1-benzyl-3-piperidinyl group, and more preferably a 1-diphenylmethyl-3-azetidinyl group.

The halogen atom in the definition of R³ may be, for example, a fluorine atom, a chlorine atom, a bromine atom, or an iodine atom, preferably a fluorine atom or a chlorine atom and more preferably a chlorine atom.

Preferably, R¹ is a methyl group, a carbamoyloxymethyl group, a 2-aminoethoxymethyl group, an ethyl group, a 2-carbamoyloxyethyl group, a 2-(2-aminoethoxy)ethyl group, an amino group or a cyano group. More preferably, R¹ is a methyl group, a carbamoyloxymethyl group, a 2-aminoethoxymethyl group, an amino group or a cyano group. Still more preferably, R¹ is a methyl group or an amino group. Most preferably, R¹ is an amino group.

Preferably, R² is a methyl group, an acetylmethyl group, a 2-tetrahydrofurylmethyl group, an ethyl group, a 2-acetylethyl group, a 2-(N-methyl-N-benzylamino)ethyl group, a 2-[N-methyl-N-(4-fluorophenylmethyl)amino]ethyl group, a 2-(N-phenyl-N-benzylamino)ethyl group, a 2-[N-(4-fluorophenyl)-N-benzylamino]ethyl group, a 2-[N-(4-fluorophenyl)-N-(4-fluorophenylmethyl)amino]ethyl group, a 2-(4-benzyl-1-piperazinyl)ethyl group, a 2-[4-(4-fluorophenylmethyl)-1-piperazinyl]ethyl group, a 2-(4-diphenylmethyl-1-piperazinyl)ethyl group, a 2-[4-(di-4-fluorophenylmethyl)-1-piperazinyl]ethyl group, an isopropyl group, an isobutyl group, a 3-phenyl-2-propenyl group, a 3-(4-fluorophenyl)-2-propenyl group, a 4-phenyl-3-butenyl group, a 2-methyl-3-phenyl-2-propenyl group, a 1-benzyl-3-azetidinyl group, a 1-diphenylmethyl-3-azetidinyl group, a 1-(di-4-fluorophenylmethyl)-3-azetidinyl group, a 1-benzyl-3-pyrrolidinyl group, a 1-(4-fluorophenylmethyl)-3-pyrrolidinyl group, a 1-diphenylmethyl-3-pyrrolidinyl group, a 1-benzyl-3-piperidinyl group, a 1-(4-fluorophenylmethyl)-3-piperidinyl group, or a 1-diphenylmethyl-3-piperidinyl group. More preferably, R² is a methyl group, an acetylmethyl group, a 2-tetrahydrofurylmethyl group, an ethyl group, a 2-(N-methyl-N-benzylamino)ethyl group, a 2-[N-methyl-N-(4-fluorophenylmethyl)amino]ethyl group, a 2-(N-phenyl-N-benzylamino)ethyl group, a 2-(4-diphenylmethyl-1-piperazinyl)ethyl group, an isopropyl group, an isobutyl group, a 3-phenyl-2-propenyl group, a 1-benzyl-3-azetidinyl group, a 1-diphenylmethyl-3-azetidinyl group, if 1-(di-4-fluorophenylmethyl)-3-azetidinyl group, a 1-benzyl-3-pyrrolidinyl group, or a 1-benzyl-3-piperidinyl group. Still more preferably, R² is a methyl group, an ethyl group, a 2-(4-diphenylmethyl-1-piperazinyl)ethyl group, an isobutyl group, a 3-phenyl-2-propenyl group, a 1-benzyl-3-azetidinyl group, a 1-diphenylmethyl-3-azetidinyl group, a 1-benzyl-3-pyrrolidinyl group, or a 1-benzyl-3-piperidinyl group. Most preferably, R² is a 1-diphenylmethyl-3-azetidinyl group.

Preferably, R³ is a 2-chlorophenyl group, a 2,3-dichlorophenyl group, a 2-nitrophenyl group, a 3-nitrophenyl group, or a 2,3-methylenedioxyphenyl group. More preferably, R³ is a 3-nitrophenyl group.

Preferably, R⁴ is a methoxycarbonyl group, an ethoxycarbonyl group, a 2-methoxyethoxycarbonyl group, an isopropoxycarbonyl group, or a 5,5-dimethyl-1,3,2-phosphorinan-2-yl group. More preferably, R⁴ is a methoxycarbonyl group, or an isopropoxycarbonyl group, and most preferably R⁴ is an isopropoxycarbonyl group.

Preferred calcium channel blockers of formula (I) are:

(1) a compound wherein R¹ is a methyl group, a carbamoyloxymethyl group, a 2-aminoethoxymethyl group, an amino group or a cyano group;

(2) a compound wherein R¹ is a methyl group, or an amino group;

(3) a compound wherein R¹ is an amino group;

(4) a compound wherein R² is a methyl group, an acetylmethyl group, a 2-tetrahydrofurylmethyl group, an ethyl group, a 2-acetylethyl group, a 2-(N-methyl-N-benzylamino)ethyl group, a 2-[N-methyl-N-(4-fluorophenylmethyl)amino]ethyl group, a 2-[N-phenyl-N-benzylamino)ethyl group, a 2-W-(4-fluorophenyl)-N-benzylamino]ethyl group, a 2-[N-(4-fluorophenyl)-N-(4-fluorophenylmethyl)amino]ethyl group, a 2-(4-benzyl-1-piperazinyl)ethyl group, a 2-[4-(4-fluorophenylmethyl)-1-piperazinyl]ethyl group, a 2-(4-diphenylmethyl-1-piperazinyl)ethyl group, a 2-[4-(di-4-fluorophenylmethyl)-1-piperazinyl]ethyl group, an isopropyl group, an isobutyl group, a 3-phenyl-2-propenyl group, a 3-(4-fluorophenyl)-2-propenyl group, a 4-phenyl-3-butenyl group, a 2-methyl-3-phenyl-2-propenyl group, a 1-benzyl-3-azetidinyl group, a 1-diphenylmethyl-3-azetidinyl group, a 1-(di-4-fluorophenylmethyl)-3-azetidinyl group, a 1-benzyl-3-pyrrolidinyl group, a 1-(4-fluorophenylmethyl)-3-pyrrolidinyl group, a 1-diphenylmethyl-3-pyrrolidinyl group, a 1-benzyl-3-piperidinyl group, a 1-(4-fluorophenylmethyl)-3-piperidinyl group, or a 1-diphenylmethyl-3-piperidinyl group;

(5) a compound wherein. R² is a methyl group, an acetylmethyl group, a 2-tetrahydrofurylmethyl group, an ethyl group, a 2-(N-methyl-N-benzylamino)ethyl group, a 2-[N-methyl-N-(4-fluorophenylmethyl)amino]ethyl group, a 2-(N-phenyl-N-benzylamino)ethyl group, a 2-(4-diphenylmethyl-1-piperazinyl)ethyl group, an isopropyl group, an isobutyl group, a 3-phenyl-2-propenyl group, a 1-benzyl-3-azetidinyl group, a 1-diphenylmethyl-3-azetidinyl group, a 1-(di-4-fluorophenylmethyl)-3-azetidinyl group, a 1-benzyl-3-pyrrolidinyl group, or a 1-benzyl-3-piperidinyl group;

(6) a compound wherein R² is a methyl group, an ethyl group, a 2-(4-diphenylmethyl-1-piperazinyl)ethyl group, an isobutyl group, a 3-phenyl-2-propenyl group, a 1-benzyl-3-azetidinyl group, a 1-diphenylmethyl-3-azetidinyl group, a 1-benzyl-3-pyrrolidinyl group, or a 1-benzyl-3-piperidinyl group;

(7) a compound wherein R² is a 1-diphenylmethyl-3-azetidinyl group;

(8) a compound wherein R³ is a 2-chlorophenyl group, a 2,3-dichlorophenyl group, a 2-nitrophenyl group, a 3-nitrophenyl group, or a 2,3-methylenedioxyphenyl group;

(9) a compound wherein R³ is a 3-nitrophenyl group;

(10) a compound wherein R⁴ is a methoxycarbonyl group, an ethoxycarbonyl group, a 2-methoxyethoxycarbonyl group, an isopropoxycarbonyl group, or a 5,5-dimethyl-1,3,2-phosphorinan-2-yl group;

(11) a compound wherein R⁴ is a methoxycarbonyl group, or an isopropoxycarbonyl group;

(12) a compound wherein R⁴ is an isopropoxycarbonyl group;

(13) a compound wherein R⁵ is a methyl group or an ethyl group; and

(14) a compound wherein R⁵ is a methyl group.

Representative calcium channel blockers of formula (I) include amlodipine, aranidipine; azelnidipine, barnidipine, benidipine, cilnidipine, efonidipine; elgodipine, felodipine, falnidipine, lemildipine, manidipine, nicardipine, nifedipine, nilvadipine, nisoldipine, nitrendipine, or pranidipine; preferably amlodipine, azelnidipine, barnidipine, benidipine, cilnidipine, felodipine, lemildipine, manidipine, nicardipine, nifedipine, nilvadipine, nisoldipine, nitrendipine, or pranidipine; more preferably amlodipine, azelnidipine, barnidipine, benidipine, manidipine, nicardipine, nisoldipine, nitrendipine, or pranidipine; still more preferably azelnidipine, barnidipine, benidipine, manidipine, or nicardipine; and most preferably azelnidipine. These calcium channel blockers of formula (I) are disclosed in U.S. Pat. No. 4,572,909, U.S. Pat. No. 4,446,325, U.S. Pat. No. 4,772,596, U.S. Pat. No. 4,220,649, U.S. Pat. No. 4,501,748, U.S. Pat. No. 4,672,068, U.S. Pat. No. 4,885,284, U.S. Pat. No. 4,952,592, U.S. Pat. No. 4,264,611, Japanese patent publication (kohyo) No. Sho 60-500255, Japanese patent publication No. Sho 59-152373, U.S. Pat. No. 4,892,875, U.S. Pat. No. 3,985,758, U.S. Pat. No. 3,485,847, U.S. Pat. No. 4,338,322, U.S. Pat. No. 4,154,839, U.S. Pat. No. 3,799,934, Japanese patent publication No. Sho 60-120861 and the like.

Planar chemical structures of these calcium channel blockers of formula (I) are shown below.

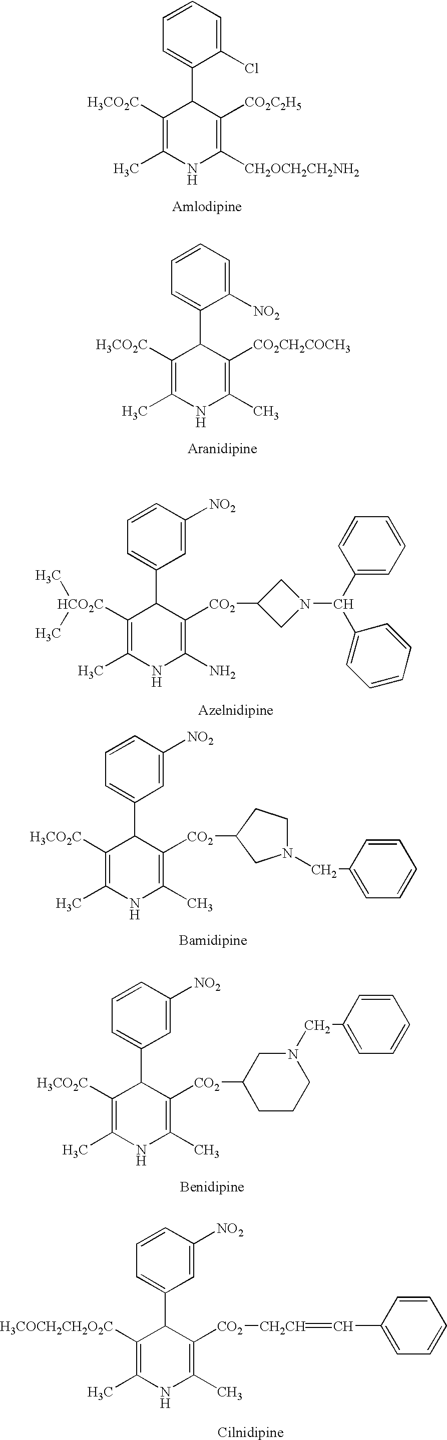

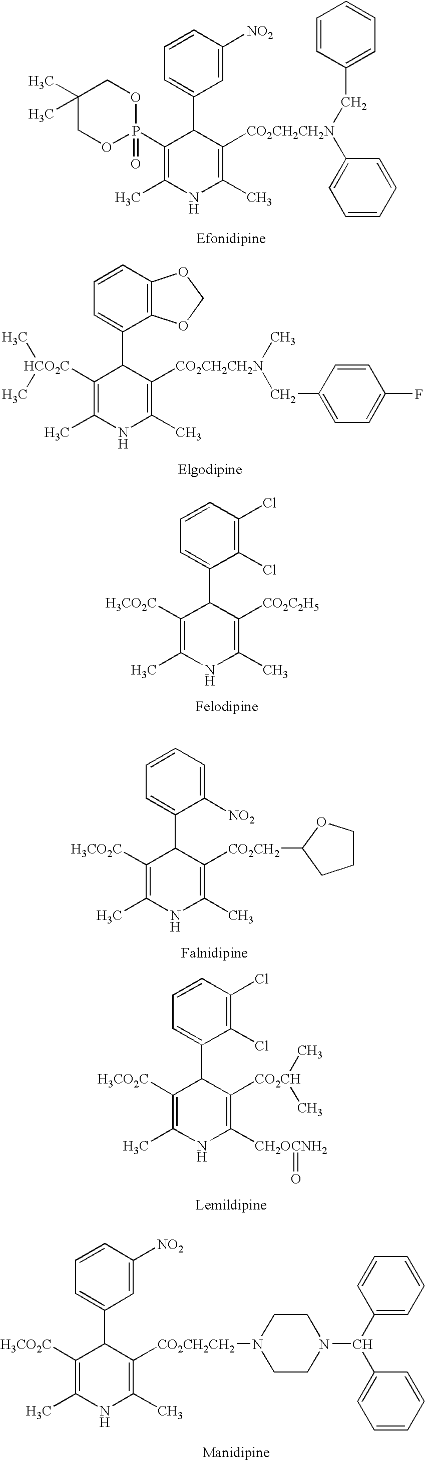

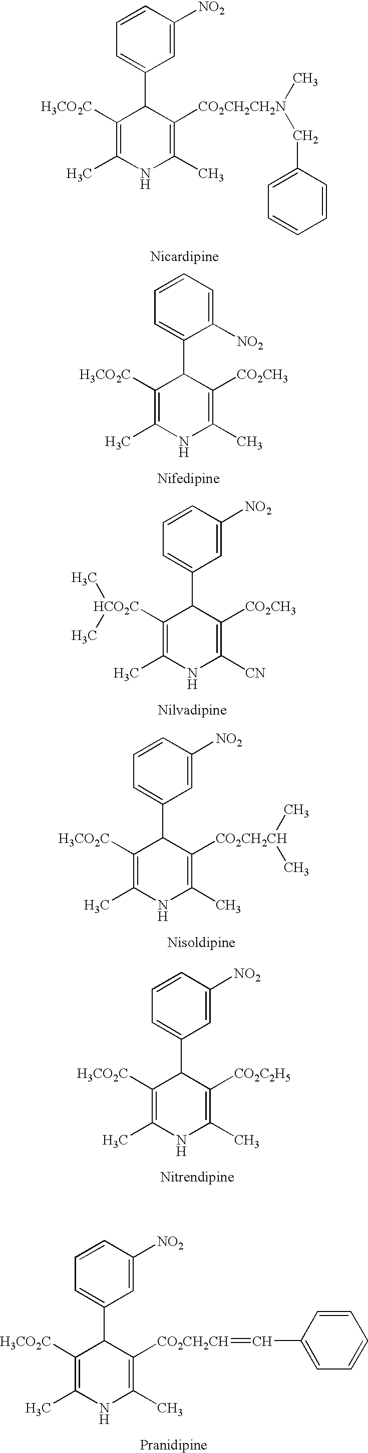

Amlodipine is 2-(2-aminoethoxymethyl)-4-(2-chlorophenyl)-3-ethoxycarbonyl-5-methoxycarbonyl-6-methyl-1,4-dihydropyridine disclosed in U.S. Pat. No. 4,572,909, Japanese patent publication No. Sho 58-167569 and the like.

Aranidipine is 3-(2-oxopropoxycarbonyl)-2,6-dimethyl-5-methoxycarbonyl-4-(2-nitrophenyl)-1,4-dihydropyridine disclosed in U.S. Pat. No. 4,446,325 and the like.

Azelnidipine is 2-amino-3-(1-diphenylmethyl-3-azetidinyloxycarbonyl)-5-isopropoxycarbonyl-6-methyl-4-(3-nitrophenyl)-1,4-dihydropyridine disclosed in U.S. Pat. No. 4,772,596, Japanese patent publication No. Sho 63-253082 and the like.

Barnidipine is 3-(1-benzyl-3-pyrrolidinyloxycarbonyl)-2,6-dimethyl-5-methoxycarbonyl-4-(3-nitrophenyl)-1,4-dihydropyridine disclosed in U.S. Pat. No. 4,220,649, Japanese patent publication No. Sho 55-301 and the like.

Benidipine is 3-(1-benzyl-3-piperidinyloxycarbonyl)-2,6-dimethyl-5-methoxycarbonyl-4-(3-nitrophenyl)-1,4-dihydropyridine and is described in the specifications of U.S. Pat. No. 4,501,748, Japanese patent publication No. Sho 59-70667 and the like.

Cilnidipine is 2,6-dimethyl-5-(2-methoxyethoxycarbonyl)-4-(3-nitrophenyl)-3-(3-phenyl-2-propenyloxycarbonyl)-1,4-dihydropyridine disclosed in U.S. Pat. No. 4,672,068, Japanese patent publication No. Sho 60-233058 and the like.

Efonidipine is 3-[2-(N-benzyl-N-phenylamino)ethoxycarbonyl]-2,6-dimethyl-5-(5,5-dimethyl-1,3,2-dioxa-2-phosphonyl)-4-(3-nitrophenyl)-1,4-dihydropyridine disclosed in U.S. Pat. No. 4,885,284, Japanese patent publication No. Sho 60-69089 and the like.

Elgodipine is 2,6-dimethyl-5-isopropoxycarbonyl-4-(2,3-methylenedioxyphenyl)-3-[2-[N-methyl-N-(4-fluorophenylmethyl)amino]ethoxycarbonyl]-1,4-dihydropyridine disclosed in U.S. Pat. No. 4,952,592, Japanese patent publication No. Hei 1-294675 and the like.

Felodipine is 3-ethoxycarbonyl-4-(2,3-dichlorophenyl)-2,6-dimethyl-5-methoxycarbonyl-1,4-dihydropyridine disclosed in U.S. Pat. No. 4,264,611, Japanese patent publication No. Sho 55-9083 and the like.

Falnidipine is 2,6-dimethyl-5-methoxycarbonyl-4-(2-nitrophenyl)-3-(2-tetrahydrofurylmethoxycarbonyl)-1,4-dihydropyridine disclosed in U.S. Pat. No. 4,656,181, Japanese patent publication (kohyo) No. Sho 60-500255 and the like.

Lemildipine is 2-carbamoyloxymethyl-4-(2,3-dichlorophenyl)-3-isopropoxycarbonyl-5-methoxycarbonyl-6-methyl-1,4-dihydropyridine disclosed in Japanese patent publication No. Sho 59-152373 and the like.

Manidipine is 2,6-dimethyl-3-[2-(4-diphenylmethyl-1-piperazinyl)ethoxycarbonyl]-5-methoxycarbonyl-4-(3-nitrophenyl)-1,4-dihydropyridine disclosed in U.S. Pat. No. 4,892,875, Japanese patent publication No. Sho 58-201765 and the like.

Nicardipine is 2,6-dimethyl-3-[2-(N-benzyl-N-methylamino)ethoxycarbonyl]-5-methoxycarbonyl-4-(3-nitrophenyl)-1,4-dihydropyridine disclosed in U.S. Pat. No. 3,985,758, Japanese patent publication No. Sho 49-108082 and the like.

Nifedipine is 2,6-dimethyl-3,5-dimethoxycarbonyl-4-(2-nitrophenyl)-1,4-dihydropyridine disclosed in U.S. Pat. No. 3,485,847 and the like.

Nilvadipine is 2-cyano-5-isopropoxycarbonyl-3-methoxycarbonyl-6-methyl-4-(3-nitrophenyl)-1,4-dihydropyridine disclosed in U.S. Pat. No. 4,338,322, Japanese patent publication No. Sho 52-5777 and the like.

Nisoldipine is 2,6-dimethyl-3-isobutoxycarbonyl-5-methoxycarbonyl-4-(3-nitrophenyl)-1,4-dihydropyridine disclosed in U.S. Pat. No. 4,154,839, Japanese patent publication No. Sho 52-59161 and the like.

Nitrendipine is 3-ethoxycarbonyl-2,6-dimethyl-5-methoxycarbonyl-4-(3-nitrophenyl)-1,4-dihydropyridine disclosed in U.S. Pat. No. 3,799,934, Japanese patent publication (after examination) No. Sho 55-27054 and the like.

Pranidipine is 2,6-dimethyl-5-methoxycarbonyl-4-(3-nitrophenyl)-3-(3-phenyl-2-propen-1-yloxycarbonyl)-1,4-dihydropyridine disclosed in U.S. Pat. No. 5,034,395, Japanese patent publication No. Sho 60-120861 and the like.

When calcium channel blockers of formula (I) have asymmetric carbon(s) and/or double bond(s), they can exist as optically active isomers, geometrical isomers and/or ring structural isomers. The present invention encompasses the individual optical, geometrical and structural isomers and mixtures thereof.

Pharmacologically acceptable salts of calcium channel blockers of formula (I) are acid addition salts, for example, hydrohalogenic acid salts such as hydrofluoride, hydrochloride, hydrobromide and hydroiodide; nitrate; perchlorate; sulfate; phosphate; carbonate; alkylsulfonates having 1 to 6 carbons optionally substituted with fluorine atom(s) such as methanesulfonates, trifluoromethanesulfonate, ethanesulfonate, pentafluoroethanesulfonate, propanesulfonate, butanesulfonate, pentanesulfonate and hexanesulfonate; arylsulfonates having 6 to 10 carbons such as benzenesulfonate and p-toluenesulfonate; carboxylic acid salts such as acetate, propionate, butyrate, benzoate, fumarate, maleate, succinate, citrate, tartrate, oxalate and malonate; or amino acid salts such as glutamate and aspartate. Preferred salts are hydrochlorides.

Calcium channel blockers of formula (I) or salts thereof can exist as hydrates and this invention encompasses such hydrates.

The pharmaceutical compositions of this invention contain 0.5 to 60 parts of a calcium channel blocker of formula (I) by weight based on 100 parts by weight of said composition, preferably 1 to 30 parts by weight.

The pharmacologically acceptable alkaline materials employed in this invention with which an aqueous solution or dispersion solution of said pharmaceutical composition can be adjusted to at least pH 8, are pharmaceutically acceptable alkaline materials known to those skilled in the art and include alkaline materials which are soluble, slightly soluble or substantially insoluble in water. Examples of such alkaline materials are alkali metal hydroxides such as lithium hydroxide, sodium hydroxide and potassium hydroxide; alkaline earth metal hydroxides such as magnesium hydroxide, calcium hydroxide and barium hydroxide; aluminium hydroxide; alkali metal carbonates such as lithium carbonate, sodium carbonate and potassium carbonate; alkaline earth metal carbonates such as magnesium carbonate, calcium carbonate and barium carbonate; alkali metal hydrogencarbonates such as lithium hydrogencarbonate, sodium bicarbonate and potassium hydrogencarbonate; di-alkali metal phosphates such as disodium phosphate and dipotassium phosphate; di-alkaline earth metal phosphates such as dimagnesium phosphate, dicalcium phosphate and dibarium phosphate; trialkali metal phosphates such as trisodium phosphate and tripotassium phosphate; alkaline earth metal oxides such as magnesium oxide and calcium oxide; aluminum oxide; alkali metal silicates such as sodium silicate and potassium silicate; Alkaline earth metal silicates such as magnesium silicate and calcium silicate; silicic acid-aluminum complex compounds such as silicic acid-alumina; aluminum-magnesium complex compounds such as magnesium aluminosilicate and magnesium aluminometasilicate; or mixtures thereof. Preferred alkaline materials are alkali metal carbonates, alkaline earth metal carbonates, alkali metal hydrogencarbonates, alkaline earth metal oxides, alkali metal silicates, aluminum-magnesium complex compounds, or mixtures thereof. More preferred alkali materials are sodium carbonate, magnesium carbonate, calcium carbonate, sodium bicarbonate, magnesium oxide, calcium oxide, magnesium silicate, calcium silicate, magnesium aluminosilicate and magnesium aluminometasilicate; or mixtures thereof. Most preferred alkali materials are sodium carbonate, sodium bicarbonate, calcium silicate, magnesium aluminosilicate and magnesium aluminometasilicate; or mixtures thereof (particularly, mixtures of sodium carbonate and magnesium aluminometasilicate aluminate or sodium bicarbonate and magnesium aluminometasilicate (in a ratio 1/20 to 1/2)).

The amount of the alkaline material is not particularly limited provided that an aqueous solution or dispersion solution of said pharmaceutical composition can be adjusted to at least pH 8 with said alkaline material. The preferred amount of the alkaline material is from 1 to 70 parts by weight based on 100 parts by weight of said composition, preferably 5 to 50 parts by weight.

The preferred pH of the aqueous solution or dispersion solution of said pharmaceutical composition is between 8 and 12, more preferably between 9 to 11. The pH of the aqueous solution or dispersion solution of said pharmaceutical composition is determined by measurement of the solution on a pH meter which solution is obtained by 1) dissolution or dispersion of a ten-fold amount of a unit dosage of said pharmaceutical composition (for example one 200 mg tablet, or one 200 mg capsule) in 100 ml of purified water as described in The Japanese Pharmacopeia (14th Edition, Official Monographs for Part II, page 1079—purified water is “water purified by distillation, ion exchange, ultrafiltration or a combination of these methods.”), 2) centrifugation of the mixture, and 3) filtration of the supernatant. Thus, a 10-fold amount of a 200 mg dosage is 2 g to be dissolved in 100 ml of purified water (or 1000 mg=1 g is dissolved in 50 ml of water as in Example 1 below).

When said pharmaceutical composition absorbs water or a small amount of water is added to said pharmaceutical composition, the pH (micro-pH) of the surroundings of the particles of said pharmaceutical composition can be adjusted to at least 8 with the pharmacologically acceptable alkaline material which is one component in this invention.

The pharmaceutical composition of this invention may appropriately contain pharmaceutically acceptable additives. Examples of such additives are excipients (for example, sugar derivatives such as lactose, sucrose, glucose, mannitol and sorbitol; starch derivatives such as corn starch, potato starch, α-starch, dextrin, carboxymethyl starch and sodium carboxymethyl starch; gelatinized starch; cellulose derivatives such as crystalline cellulose, methylcellulose, hydroxypropylcellulose, lower substituted hydroxypropylcellulose, hydroxypropylmethylcellulose, carboxymethylcellulose, calcium carboxymethylcellulose, cross-linked carboxymethylcellulose and cross-linked sodium carboxymethylcellulose; acacia; dextran; pullulan; silicate derivatives such as light silicic acid anhydride, silicic acid hydrate, synthetic aluminum silicate and magnesium aluminometasilicate; phosphate derivatives such as dicalcium phosphate; chloride salt derivatives such as sodium chloride; carbonate derivatives such as calcium carbonate; sulfate derivatives such as calcium sulfate; or mixtures thereof; preferably sugar derivatives, cellulose derivatives or mixtures thereof, more preferably mannitol, crystalline cellulose or mixtures thereof), binding agents (for example, compounds illustrated above as excipients, gelatin, polyvinylpyrrolidone, macrogol, or mixtures thereof; preferably cellulose derivatives or mixtures thereof; more preferably hydroxypropylmethyl cellulose), disintegrating agents (for example, the compounds illustrated above as excipients; cross-linked polyvinylpyrrolidone; or mixtures thereof; preferably cellulose derivatives or mixtures thereof; more preferably lower substituted hydroxypropylmethylcellulose, calcium carboxymethylcellulose or mixtures thereof), lubricating agents (for example, stearic acid; metal stearates such as calcium stearate and magnesium stearate; metal benzoates such as sodium benzoate; waxes such as beeswax and spermaceti; boric acid; glycol; carboxylic acids such as fumaric acid and adipic acid; metal sulfates such as sodium sulfate; Leucine; metal lauryl sulfates such as sodium lauryl sulfate and magnesium lauryl sulfate; the silicate derivatives illustrated above as excipients; the cellulose derivatives illustrated above as excipients; hydrogenated vegetable oil; carnauba wax; sucrose esters of fatty acids; or mixtures thereof; preferably metal stearates, silicate derivatives, or mixtures thereof and more preferably calcium stearate, magnesium stearate, silicic acid anhydride, or mixtures thereof), stabilizing agents (for example, benzoic acid, metal benzoates such as sodium benzoate; paraoxybenzoates such as methylparaben and propylparaben; alcohols such as chlorobutanol, benzyl alcohol and phenylethyl alcohol; benzalkonium chloride; phenol derivatives such as phenol or cresol; thimerosal; acetic anhydride; sorbic acid or mixtures thereof; preferably metal benzoates, paraoxybenzoates, or mixtures thereof; more preferably sodium benzoate, methylparaben, propylparaben, or mixtures thereof), fluidizing agents (for example, the silicate derivatives illustrated above as excipients; talc; or mixtures thereof; preferably light silicic acid anhydride, talc or mixtures thereof), surface activating agents (for example, polysorbates such as polysorbate 80; polyoxyethylene hydrogenated castol oils such as polyoxyethylene hydrogenated castol oil 60; sorbitan esters of fatty acids; sucrose esters of fatty acids; polyoxyethylenepolyoxypropylenglycols; polyoxyethylene ethers of fatty acids; polyoxyl stearates; or mixtures thereof; preferably polysorbate 80, polyoxyethylene hydrogenated castol oil 60 or mixtures thereof), coloring agents, anti-oxidating agents, corrigents (for example, sweetening, souring and flavoring agents which are conventionally used), or diluents.

Additives employed in this invention and the amount of said additives will vary with tablets, capsules, and other dosage forms, and they can be determined by techniques known to those skilled in the art. Tablets may usually contain binder(s) in an amount of 1 to 10 parts by weight (preferably 3 to 5 parts), disintegrant(s) in an amount of 1 to 40 parts by weight (preferably 5 to 30 parts), lubricant(s) in an amount of 0.1 to 10 parts by weight (preferably 0.5 to 3 parts) and fluidizing agent(s) in an amount of 1 to 10 parts by weight (preferably 2 to 5) based on 100 parts by weight of said pharmaceutical composition.

The calcium channel blockers of formula (I), which are active ingredients of this invention, are known compounds or can be easily prepared according to techniques known to those skilled in the art (for example U.S. Pat. No. 4,572,909, U.S. Pat. No. 4,446,325, U.S. Pat. No. 4,772,596, Japanese patent publication No. Sho 63-253082, U.S. Pat. No. 4,220,649, U.S. Pat. No. 4,501,748, U.S. Pat. No. 4,672,068, U.S. Pat. No. 4,885,284, U.S. Pat. No. 4,952,592, U.S. Pat. No. 4,264,611, Japanese patent publication (kohyo) No. Sho 60-500255, Japanese patent publication No. Sho 59-152373, U.S. Pat. No. 4,892,875, U.S. Pat. No. 3,985,758, U.S. Pat. No. 3,485,847, U.S. Pat. No. 4,338,322, U.S. Pat. No. 4,154,839, U.S. Pat. No. 3,799,934, Japanese patent publication No. Sho 60-120861, and the like).

The pharmaceutical compositions of the present invention can be prepared easily by using calcium channel blockers of formula (I) or salts thereof, alkaline materials and pharmaceutically acceptable additives in a known manner (for example, procedures such as mixing and kneading with water and wet granulation, etc.). Formulations such as tablets, capsules and granules, for example, can be prepared as follows. To the alkaline materials placed in a high shear granulator is added surfactant(s) as needed, and then a calcium channel blocker of formula (I) or a salt thereof, fillers, binders and disintegrants are furthermore added with mixing. In some cases, other kinds of alkaline materials are also added as needed. Subsequently, an aqueous solution of the binder(s) is added to the mixture obtained to prepare a wet mass in the high shear granulator. In the preparation of tablets and capsules, the wet mass obtained is dried in a fluid bed dryer, and the dried mass obtained is cut by a cutting mill and passed through a screen. The desired tablets or capsules can be prepared by mixing the screened granules and lubricant(s) with a V-shaped blender and then tableting or filling the resulting mixture into capsules, respectively. On the other hand, in the preparation of granules, the wet mass obtained above is extruded using an extrusion granulator to prepare wet granules, which are then dried using an air-through tray dryer. The desired granules can be obtained by cutting the dried granules obtained using the cutting mill and then passing through a screen.

The present invention is described in more detail by Examples, but the present invention is not limited to these Examples.

### Example 1

### Tablets 1

The desired tablets were prepared using the components, the quantity of each of which is listed in the formula shown in Table 1, as follows.

To light magnesium aluminometasilicate (Grade FL2) placed in a high shear granulator was added polysorbate 80 with stirring, and then Azelnidipine, crystalline cellulose, D-mannitol, low substituted hydroxypropylcellulose and sodium bicarbonate were added successively with mixing. Subsequently, an aqueous hydroxypropylcellulose solution was added to the mixture to prepare a wet mass, which was dried in a fluid bed dryer into which inlet air at 90° C. was supplied continuously until the temperature of the exhausted air from the dryer went up to 55° C. The dried mass obtained was cut by a cutting mill and passed through a screen of 1.0-mm meshes. The desired tablets were prepared by mixing the screened granules and magnesium stearate for 10 min using a V-shaped blender and then compressing the resulting mixture using a tableting machine with a punch of 8.0-mm diameter.

In each of Examples 1-5 and Reference example 1, 8 mg of Azelnidipine was used

TABLE 1

| | Quantity |
|---|---|
| Component | (Weight percentage) |
| Azelnidipine | 5 |
| Crystalline cellulose | 5 |
| D-mannitol | 8 |
| Low substituted hydroxypropylcellulose | 15 |
| Light magnesium aluminometasilicate | 45 |
| Sodium bicarbonate | 3 |
| Hydroxypropylcellulose | 3 |
| Polysorbate 80 | 15 |
| Magnesium stearate | 1 |
| Total | 100 |

This formulation was pulverized in an agate mortar and passed through a sieve with 20 meshes. Subsequently, 1000 mg of the pulverized formulation obtained (corresponding to five tablets) was placed in a centrifuge tube and after the addition of 50 ml of purified water as defined by The Pharmacopoeia of Japan, the resulting mixture was shaken for 20 min using a shaker. After shaking, the resulting suspension was centrifuged at 3000 rpm for 10 min and the supernatant obtained was passed through a filter with a pore size of 0.45-μm, and then the pH value of the filtrate was measured with a pH meter. The pH value of the solution obtained was 9.5.

When this formulation was stored at 25° C. under lightproof and water-resistant conditions, 98% of the active ingredient in this formulation was detected as unaltered even after storage for 36 months.

### Example 2

### Tablets 2

The desired tablets were prepared using the components, the quantity of each of which is listed in the formula shown in Table 2, as follows.

To a mixture of light magnesium aluminometasilicate (Grade FL2) and light silicic acid anhydride in a high shear granulator was added polysorbate 80 with stirring, and then Azelnidipine, crystalline cellulose, D-mannitol, low substituted hydroxypropylcellulose, carboxymethylcellulose calcium (carmellose calcium) and sodium bicarbonate were added successively with mixing. Subsequently, an aqueous hydroxypropylcellulose solution was added to the mixture to prepare a wet mass, which was dried in a fluid bed dryer into which inlet air at 90° C. was supplied continuously until the temperature of the exhausted air from the dryer went up to 55° C. The dried mass obtained was cut by a cutting mill and passed through a screen of 1.0-mm meshes. The desired tablets were prepared by mixing the screened granules and magnesium stearate for 10 min using a V-shaped blender and then compressing the resulting mixture using a tableting machine with a punch of 8.0-mm diameter.

TABLE 2

| | Quantity |
|---|---|
| Component | (Weight percentage) |
| Azelnidipine | 5 |
| Crystalline cellulose | 5 |
| D-mannitol | 15 |
| Low substituted hydroxypropylcellulose | 15 |
| Carmellose calcium | 6 |
| Light magnesium aluminometasilicate | 25 |
| Light silicic acid anhydride | 6 |
| Sodium bicarbonate | 5 |
| Hydroxypropylcellulose | 5 |
| Polysorbate 80 | 12 |
| Magnesium stearate | 1 |
| Total | 100 |

The pH value of this formulation was measured in a similar manner to that mentioned in Example 1 The pH value of the solution obtained was 10.0.

When this formulation was stored at 25° C. under lightproof and water-resistant conditions, 99% of the active ingredient in this formulation was detected as unaltered even after storage for 36 months.

### Example 3

### Capsules 1

The desired capsules were obtained by preparing a mixture of components, the quantity of each of which is listed in the formula shown in Table 2, in a similar manner to that mentioned in Example 2 and then filling a defined amount of the resulting mixture into each No. 3 capsule.

The pH value of this formulation was measured in a similar manner to that mentioned in Example 1. The pH value of the solution obtained was 10.0.

When this formulation was stored at 25° C. under lightproof and water-resistant conditions, 98% of the active ingredient in this formulation was detected as unaltered even after storage for 36 months.

### Example 4

### Tablets 3

The desired tablets were prepared using sodium carbonate instead of sodium bicarbonate listed in the formula in Table 2 in a similar manner to that mentioned in Example 2.

The pH value of this formulation was measured in a similar manner to that mentioned in Example 1. The pH value of the solution obtained was 11.0.

When this formulation was stored at 25° C. under lightproof and water-resistant conditions, 95% of the active ingredient in this formulation was detected as unaltered even after storage for 36 months.

### Example 5

### Tablets 4

The desired tablets were prepared using the components, the quantity of each of which is listed in the formula shown in Table 3, as follows.

To calcium silicate placed in a high shear granulator was added polysorbate 80 with stirring, and then Azelnidipine, D-mannitol and low substituted hydroxypropylcellulose were added successively with mixing. Subsequently, an aqueous hydroxypropylcellulose solution was added to the mixture to prepare a wet mass, which was dried in a fluid bed dryer into which inlet air at 90° C. was supplied continuously until the temperature of the exhausted air from the dryer went up to 55° C. The dried mass obtained was cut by a cutting mill and passed through a screen of 1.0-mm meshes. The desired tablets were prepared by mixing the screened granules and magnesium stearate for 10 min with a V-shaped blender and then compressing the resulting mixture using a tableting machine with a punch of 8.0-mm diameter.

TABLE 3

| | Quantity |
|---|---|
| Component | (Weight percentage) |
| Azelnidipine | 5 |
| D-mannitol | 34 |
| Low substituted hydroxypropylcellulose | 20 |
| Calcium silicate | 20 |
| Hydroxypropylcellulose | 5 |
| Polysorbate 80 | 15 |
| Magnesium stearate | 1 |
| Total | 100 |

The pH value of this formulation was measured in a similar manner to that mentioned in Example 1. The pH value of the solution obtained was 9.3.

When this formulation was stored at 25° C. under lightproof and water-resistant conditions, 97% of the active ingredient in this formulation was detected as unaltered even after storage for 36 months.

### Reference Example 1

### Tablets A

The desired tablets were prepared using the components, the quantity of each of which is listed in the formula shown in Table 4, as follows.

Azelnidipine, D-mannitol and low substituted hydroxypropylcellulose were mixed in a high shear granulator, and then polysorbate 80 was further added with mixing. Subsequently, an aqueous hydroxypropylcellulose solution was added to the mixture to prepare a wet mass, which was dried in a fluid bed dryer into which inlet air at 90° C. was supplied continuously until the temperature of the exhausted air from the dryer went up to 55° C. The dried mass obtained was cut by a cutting mill and passed through a screen of 1.0-mm meshes. The desired tablets were prepared by mixing the screened granules and magnesium stearate for 10 min with a V-shaped blender and then compressing the resulting mixture using a tableting machine with a punch of 8.0-mm diameter.

TABLE 4

| | Quantity |
|---|---|
| Component | (Weight percentage) |
| Azelnidipine | 5 |
| D-mannitol | 57 |
| Low substituted hydroxypropylcellulose | 20 |
| Hydroxypropylcellulose | 5 |
| Polysorbate 80 | 12 |
| Magnesium stearate | 1 |
| Total | 100 |

The pH value of this formulation was measured in a similar manner to that mentioned in Example 1. The pH value of the solution obtained was 7.4.

When this formulation was stored at 25° C. under lightproof and water-resistant conditions, 70% of the active ingredient in this formulation was detected as unaltered after storage for 36 months.

The pharmaceutical compositions of this invention exhibit excellent storage stability, rapid absorption through the intestinal tract and can be prepared by an easy wet granulation method. These pharmaceutical compositions, therefore, are useful compositions as a medical formulation.

What is claimed is:

1. A pharmaceutical composition containing a calcium channel blocker wherein the calcium channel blocker is azelnidipine or a pharmacologically acceptable salt thereof and a pharmacologically acceptable alkaline material which is added to an extent such that an aqueous solution or dispersion solution of said pharmaceutical composition containing a calcium channel blocker has a pH of at least 8.

2. A pharmaceutical composition according to claim 1 wherein the alkaline material is an alkali metal carbonate, an alkaline earth metal carbonate, an alkali metal hydrogencarbonate, an alkaline earth metal oxide, an alkali metal silicate, an alkaline earth metal silicate, an aluminum-magnesium complex compound, or a mixture thereof.

3. A pharmaceutical composition according to claim 1 wherein the alkaline material is sodium carbonate, magnesium carbonate, calcium carbonate, sodium bicarbonate, magnesium oxide, calcium oxide, magnesium silicate, calcium silicate, magnesium aluminosilicate, magnesium aluminometasilicate, or a mixture thereof.

4. A pharmaceutical composition according to claim 1 wherein the alkaline material is a mixture of sodium carbonate and magnesium aluminometasilicate, or a mixture of sodium bicarbonate and magnesium aluminometasilicate.

5. A pharmaceutical composition according to claim 1 wherein the alkaline material is an alkali metal hydroxide, an alkaline earth metal hydroxide, an aluminum hydroxide, an alkali metal carbonate, an alkaline earth metal carbonate, an alkali metal hydrogencarbonate, a di-alkali metal phosphate, a di-alkaline earth metal phosphate, a tri-alkali metal phosphate, an alkaline earth metal oxide, aluminum oxide, an alkali metal silicate, an alkaline earth metal silicate, a silicic acid-aluminum complex compound, an aluminum-magnesium complex compound, or a mixture thereof.

6. A pharmaceutical composition according to claim 1 wherein the alkaline material is sodium carbonate, sodium bicarbonate, calcium silicate, magnesium aluminosilicate, magnesium aluminometasilicate, or a mixture thereof.

7. A pharmaceutical composition according to any one of claims 1, 2, 3, 4 and 5 wherein the pH of an aqueous solution or dispersion solution of said pharmaceutical composition is between 8 and 12.

8. A pharmaceutical composition according to any one of claims 1, 2, 3, 4 and 5 wherein the pH of an aqueous solution or dispersion solution of said pharmaceutical composition is between 9 and 11.

9. A pharmaceutical composition according to any one of claims 1, 2, 3, 4 and 5 wherein the pH of an aqueous solution or dispersion solution of said pharmaceutical composition is between 9.3 and 11.

10. A pharmaceutical composition according to claim 7 wherein the pharmaceutical composition is a tablet.

11. A pharmaceutical composition according to claim 8 wherein the pharmaceutical composition is a tablet.
